(19) United States
(12) Patent Application Publication
(10) Pub. No.: US 2014/0082957 A1
(43) Pub. Date: Mar. 27, 2014

(54) Attachment to air moving device or system for the purpose of scenting spaces

(71) Applicant: Neal F. Harris, Los Angeles, CA (US)

(72) Inventor: Neal F. Harris, Los Angeles, CA (US)

(21) Appl. No.: 13/573,591

(22) Filed: Sep. 27, 2012

(51) Int. Cl. A61L 9/04

(52) U.S. Cl. 34/239

(57) ABSTRACT

An attachment for a fan, blower of other air moving device encompassing, and made of or designed to hold a scented material. The attachment may be constructed from any flexible, rigid or semi rigid material. A scented material base utilizing or made of paper, polymer, plastic, cardboard, ceramic, gel or other fragrance containing substrate that can be contained or placed inside said attachment. When the fan or blower is turned on a scent is emitted throughout the space. The attachment is available in standard or custom sizes, scents, shapes or colors to fit any standard fan or blower. The air movement produced creates a scent dispersion/diffusion effect.

### CROSS REFERENCE TO RELATED APPLICATION

This continuation patent application claims priority to the non-provisional patent application having Ser. No. 12/798,145, filed on Mar. 30, 2010, which claims priority to the provisional patent application having Ser. No. 61/211,643, filed on Apr. 1, 2009.

### FIELD OF THE INVENTION

An attachment for a fan, blower or other air moving device which includes a flexible, rigid, or semi rigid material that can hold a scented composition, whether it is of a gel, liquid, pellets, or other fragrance, so that when the fan or blower is turned on, a scent is emitted throughout the space during operations of the air moving means.

### BACKGROUND OF THE INVENTION

Over the years, there have been many inventions and attempts made to dispense fragrances that deal with fans, lights, electrical plugs, heater diffusers or elaborate scent emitting devices. Many of these inventions are elaborate, even motor driven, battery operated, complicated and costly devices. Many of these devices work well but require the consumer to purchase “refill” scent cartridges from the original manufacturer, the consumer has no flexibility once they begin to use the particular system. This invention offers the end user flexibility in the design and scent they choose to use. The attachment can be custom designed to fit on any standard fan or blower offering the consumer the opportunity to reuse current systems while avoiding costly investment in completely new equipment. The attachment can be designed to fit on many standard fans or blowers readily available to consumers by retailers or wholesalers. Consumers may utilize their preferred scents or choose from a library of standard or custom scents. The consumer is offered flexibility and a wide array of choices of scent without being “locked into” a particular brand or specific scent dispersion system.

### SUMMARY OF THE INVENTION

This invention relates to an attachment to an air moving device, whether it be any type of packaging, a fragrance holding substrate, such as paper, cardboard, ceramic, cloth, pellets, or the like, that may be attached to a fan or blower, such that once the air moving device is turned on, the scent will be disbursed and defused throughout the room in which the fan or blower locates.

The invention includes any type of a shaped support, whether it is a round, square, rectangular, oval, or other shaped attachment, that may be connected in front, inside, or behind an air movement source. The attachment may be secured to the air moving source by means of any type of fastening, so that it is taped, such as a double faced adhesive tape, an adhesive, any type of a frame that may clip, hook, tie or secure by other means of attachment, or even be Velcroed, to the air moving device. The attachment may include means for holding of a fragrance, and it is used to defuse the scent as the air moves across the fragrant material, during its passage into a building, room, or the like. The attachment will be located at a position where air may be forced to cross its scented material, or pulled across the same, so as to provide either a push, or pull, of the fragrance, and for its emission into a room, to be scented.

The actual structure of the device may include a perforated or screened type of material, whether it be wire screen, wire cloth, a threaded screen material, or any other type of perforated screen that will allow for the passage of air therethrough, and its impingement upon the scented material, such as scent absorbed or coated pellets, or beads, as can be noted. The screen material, which may be provided at both the front side and the back side of the attachment, may be adhesively adhered or heat sealed at its side, top and bottom edges, or it may be heat sealed together where a polymer screen may be used for this purpose, or it can have a frame work around its perimeter, to hold the scented pellets in place. In addition, fastening means may be provided at the upper and lower edges of the attachment, and whether it be of mechanical fastener, Velcro, or any other means for fastening of the attachment to usually the grid work of the fan or blower, or where this device may be formed more as a filter, as shown in FIG. 4, it may have a paper border formed around its periphery, so that it can be applied into the blower of a furnace, air conditioner, or the like, during usage, and emit a scented fragrance into the room, or rooms, where the blower disperses its moving air.

The air flowing means may include any type of an air moving device, such as a fan, blower, air handling system, or any type of an inflatable device. The attachment may include a series or plurality of aeration holes, throughout its shape, so that the air is free to slowly pass through the apertures, or foraminous material and pick up the fragrance, to enhance its evaporation into the room, and to control the fragrance assimilation into the air, passing into the room, to provide for its enhancement in the aroma of the air present in the room, or other building structure, during usage.

The insert is presented with a fragrance formulation, and in the specific application, the formulations applied to the attachment, or insert, at a load of about 0.09 gm/in². The attachment may include evaporation enhancers, and such evaporation enhancers include denatured alcohol (39c). The device may also include evaporation retardants, which may include one of dipropylene glycol (DPG), diethyl phthalate (DEP), or benzyl benzoate (BB). The various types of fragrances that are readily available in the art can be used in conjunction with the formation of the attachment of this invention, and these fragrances are well known in the art of room air fresheners, and related devices and technology.

The attachment may be made into a decorative motif, as where it may be applied onto the return air duct, in a room, of the blower system. To aid in the packaging of the insert or attachment for its shipment of and marketing, it may include an enclosing film, screen, or other material, that can keep the fragrance attachment intact, while it is displayed for marketing, or before its usages, and then such film can be removed to expose the fragrance to the passing air during its application and usage.

It is, therefore, the principal object of this invention to provide an attachment for application to an air vent, either upstream or downstream from a blower, so that its fragrance insert can be picked up by the moving air, and transmitted into a room, building, or the like, to enhance its aroma.

Still another object of this invention is to provide a fragrance insert, supported by an attachment that may be conveniently and easily applied to and within the path of moving air, to allow for the pick up of its fragrance, and transfer into an occupied space, to enhance its aroma.

Still another object of this invention is to provide a packaged attachment, including a fragrance insert, that can be conveniently packaged, shipped, stored, and displayed for marketing; until such time as it is purchased and applied by the home or building owner, as during usage.

These and other objects may become more apparent to those skilled in the art upon review of the summary of the invention as provided herein, and upon undertaking a study of the description of its preferred embodiment, in view of the drawings.

DESCRIPTION OF THE DRAWINGS

In referring to the drawings:

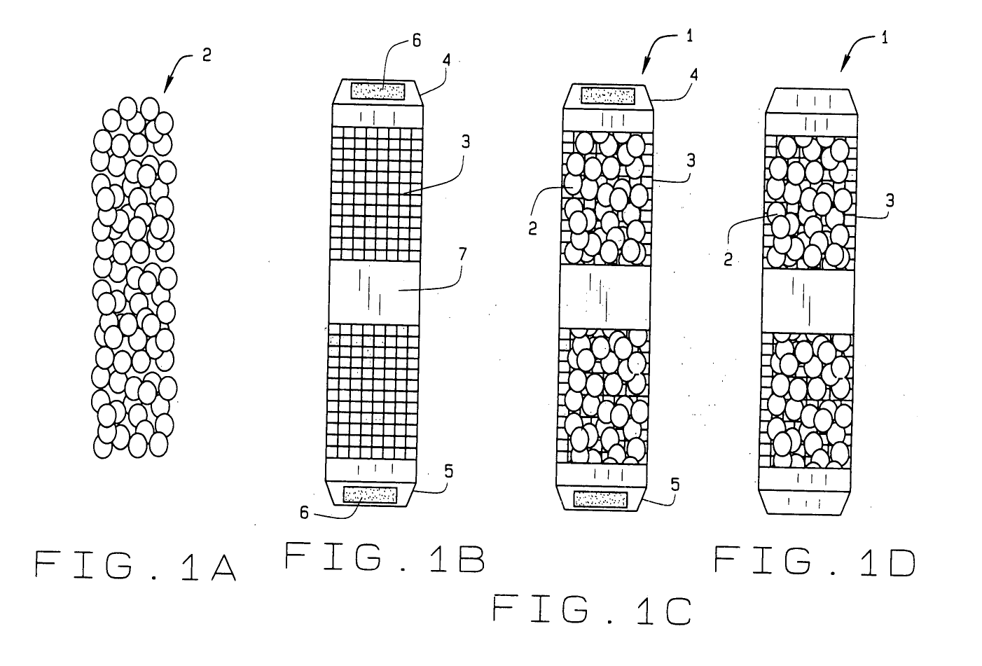

FIG. 1A provides a display of the loose scent beads or pellets before their insertion into the strip holder attachment of this invention;

FIG. 1B shows a front view of the attachment formed as a flexible mesh strip that can be strapped or otherwise fastened to a fan, blower, vent system, or the like;

FIG. 1C shows the completed flexible strip attachment from the front, with the back side being of similar appearance, but in this figure the front screen has been removed to show the fragrance pellets;

FIG. 1D shows the back view of the attachment, with the back screen being removed to disclose the fragrance pellets;

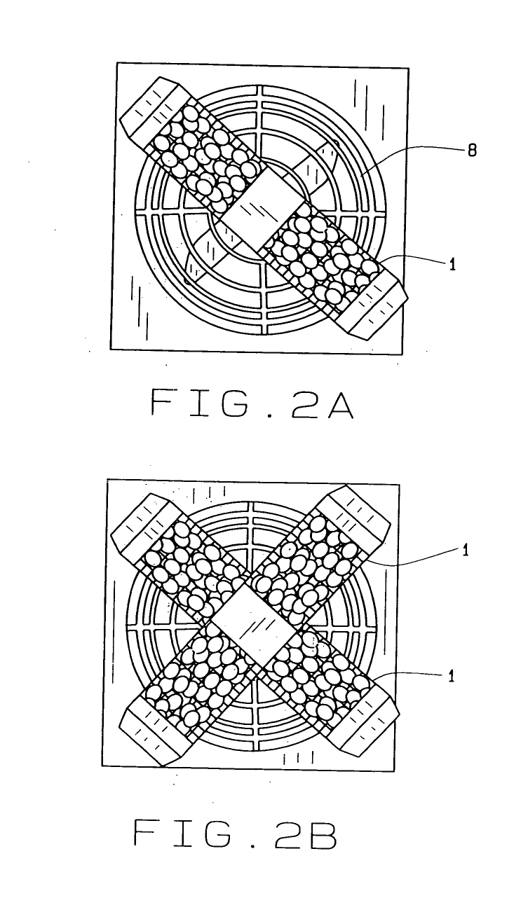

FIG. 2A shows the connection of one of the attachments to a fan;

FIG. 2B shows the connection of a plurality of attachments to a fan;

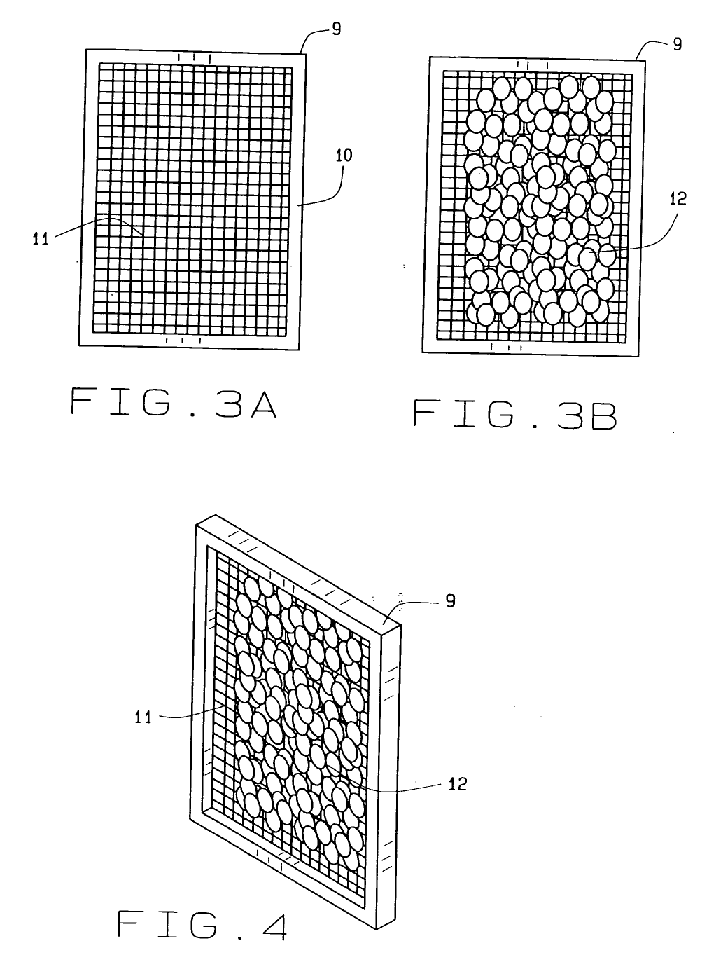

FIG. 3A shows an attachment, formed as a filter, during its assembly;

FIG. 3B shows the attachment, as a filter, with the scented pellets being applied thereon; and

FIG. 4 shows an isometric view of the attachment filter of FIGS. 3A and 3B, in its fully assembled stage, with the exception that the screen material has been removed from the front to show the arrangement of the fragrance pellets.

### DESCRIPTION OF THE PREFERRED EMBODIMENT

In referring to the drawings, and in particular FIGS. 1A through 1D, the assembly of the attachment 1 of this invention is readily disclosed. In FIG. 1A, the various pellets that are coated with a fragrance, or into which a fragrance is absorbed, are shown at 2. These can be any type of pellet that can absorb or be coated with a fragrance, such as ceramic, wood chips, paperboard, or any other type of pellet that can function as a carrier and absorb a fragrance which can be emitted as a stream of air is passed thereby, as being generated from a blower, fan, or the like. As noted in FIG. 1B, the attachment, in this embodiment, includes a strip of a foraminous cloth 3 whether it be wire cloth, a textile screen, polymer screen, or any related type of material that will hold the pellets 2 in place, within the interior of the attachment, but at the same time, allow the free flow of air therethrough. The attachment has upper and lower ends, as at 4 and 5, and these may be formed of polymer, paperboard, or the like, that provides for a seal off of the ends of the wire cloth package, as at 3, to hold the scented pellets therein. In addition, any type of fastener, such as noted at 6, whether it be Velcro, a mechanical fastener, or any other means for connection of the attachment to the grid work of a blower, fan, or the like, is provided. A reinforcing strip, as at 7, which may also be formed of plastic, paperboard, metal, or the like, is provided at the midpoint, to add stability to the attachment, since it may have some length. These attachments may have a length up to a foot, more or less, in order to provide adequate surface area upon which the fragrance can be applied, in preparation for usage of the attachment for its intended purposes.

FIG. 1C shows the attachment in the state of its formation, but the front surface of the wire screen 3 has been removed, for disclosure purposes. Nevertheless, it can be seen how the fragrance pellets are spread evenly about the length of the attachment, in its formation. FIG. 1D shows a back view of the attachment.

The sides of the attachment, particularly where it may be formed of two sheets of wire screen material, may be either adhesively connected together, heat sealed along the edges, stitched in place to provide for closure, or where the foraminous screen may be formed of one piece, it simply may be folded over, to form the front and back sides of the attachment, and then be connected along its free edges, to provide for their closure.

FIG. 2A shows how the attachment 1 of this invention may be applied onto the screen 8 of the shown blower, fan, or exhaust fan. FIG. 2B shows how a pair of the attachments 1 can be applied in a cross pattern upon the screen of the blower, as noted.

As to be noted in FIGS. 3A, 3B, and FIG. 4, the attachment is formed similar to a filter, such as a air conditioner or furnace filter, and which may be fabricated of a variety of materials, whether it be plastic, paper, cardboard, paperboard, even metal, or any other type of support means that may be able to hold the components of this invention in place in preparation and during usage. The attachment is shown specifically at 9, and on the back surface, may include any type of fastener, such as Velcro, mechanical fastener, or the like, to hold the attachment in place. Even a double faced adhesive, or an adhesive, tape, or any type of clip, or other attachment means, for holding the frame 9 of the attachment in place, can be used. The framework 10 may be similar to other filters, having, for example, a paperboard framework, usually formed in a U-shape configuration, in order to support the foraminous screen 11, throughout its perimeter, within the framework, for forming the attachment 9. FIG. 3B shows how a plurality of the scented pellets 12 are spread out upon the screen material 11, and then to complete the assembly of the attachment, another foraminous screen “not shown” may be applied to the upper surface, in order to embrace and hold the various scented pellets therein, and evenly spread, upon completion of the attachment 9. FIG. 4 shows the completed assembly, but once again, the foraminous screen forming the front surface, overlying the scented pellets or beads, as ready for application to a furnace, air conditioner, and to function not only as a filter, but as means for dissemination of a fragrance into a room, home, building, or the like, during usage and application of this invention.

The fragrance formulation that is applied to the beads or pellets, of the type as previously described, is normally that of a perfume like composition, and normally applied to these beads in liquid form. But, in this instance, as summarized, the fragrance may include several of evaporation modifiers, enhancers, or retardants, in order to control the rate of evaporation of the scented material during the process of its usage, and this depends upon the fragrance formulation, to achieve a sustained release of the scent. The fragrance may further include an enhancer, as in the nature of a denatured alcohol, which may even speed up the evaporation of the fragrance, as the air passes over it. In addition, the formulation may include evaporation retardants, that allows for the controlled dissipation of the fragrance into the ambient air, and such evaporation retardants may include dipropylene glycol (DPG), diethyl phthalate (DEP), or benzyl benzoate (BB), as examples.

In order to enhance the preservation of the fragrance, as the attachment unit is being manufactured, shipped, stored, or displayed in a market place for sale, and before the purchaser makes use of it, a thin film “not shown” may be applied upon the front and back surfaces of the attachment, and such film may be of any type of polymer film, such as polyethylene, or the like, that can be peeled free from the perimeter frame, once the unit is ready for application and usage.

Variations or modifications to the subject matter of this invention may occur to those skilled in the art upon review of this development as provided herein. Such variations, if within the spirit and scope of this invention, are intended to the encompassed within the scope of any claims to patent protection provided herein. The description of the invention, and its depiction in the drawings, is primarily set forth for illustrative purposes only.

I claim:

1. A fragrance material disseminating attachment comprising one of a cross, round, square, rectangular, oval, strip configuration, or custom configured or other shaped attachment, being shaped in the format of a decorative motif, and which may be attached in front, inside, or behind an air movement source, said attachment incorporating a fragrant material, said fragrant material including evaporation modifiers, enhances, or retardants to control the evaporation process, said evaporation enhancer including denatured alcohol and wherein said fragrant material which includes an evaporation retardant includes one of dipropylene glycol (DPG), diethyl phthalate (DEP) and benzyl benzoate (BB), said attachment being affixed using one of Velcro, tape, clip, hook, tie or other means of attachment, said attachment diffuses fragrance scent as air moves through the fragrant material, and air may be forced across the fragrant material through one of pushed air or pulling of the air through said fragrant material, and the air flow may be created by one of an air moving device, fan, blower, air handling system, and inflatable device, said attachment includes a plurality of holes throughout the shape of said attachment, with the holes allowing air to flow freely through the attachment for improving the evaporation rate and controlling the fragrance dissemination into the ambient air, said attachment having a surface that is presented with the fragrant formulation, the fragrant formulation is applied to pellets or beads and said fragrance treated pellets or beads applied onto the surface of the attachment at a load of about 0.09 gm/in², said attachment having a film, screen, or other porous material being applied over the attachment surface and its pellets or beads after the fragrance has been applied to the said attachment surface, said film being made of a material through which the fragrance can pass, and said film controlling the evaporation process of the fragrance from the attachment, said attachment being made of one of paper, gel, ceramic, porcelain, wooden, non-woven, porous, natural or other synthetic material that holds and allows the evaporative remittance of said fragrance material, and said attachment includes a perimeter framework, said framework having front and back foraminous material surfaces so that the fragrant treated pellets or beads intermediate the front and back foraminous surfaces holding fragrance treated pellets or beads in place during usage and application.

2. The insert of claim 1 wherein the film comprises plasticizing agents.

3. The attachment of claim 1 wherein the fragrant material includes microencapsulated fragrance oil.

4. The insert of claim 1 wherein the attachment is constructed of one of extruded polyethylene, molded polystyrene, or polymer based fragrance materials.

5. The insert of claim 1 wherein the attachment is constructed of a ceramic, clay, gel or similar based material that holds and allows evaporative emittance of the fragrance.

6. The fragrance emitting attachment of claim 1 wherein the fragrant material includes said scented beads that have been coated or absorbed with a fragrance.

7. The fragrant attachment of claim 1 wherein the fragrant material includes pellets that have been coated or absorbed with the fragrant material.

8. The fragrant attachment of claim 1 wherein the attachment is fabricated in strip form, the strip including a front and back surface of foraminous material, the fragrant material comprising said series of beads or pellets that are coated or absorbent of a fragrance, and which are located within the front and back surfaces of the foraminous material during assembly.

9. The fragrant attachment of claim 8 wherein the outer perimeter edges of the foraminous material forming the front and back surfaces of the strip are sealed to contain the fragrance treated beads or pellets within the attachment.
